(19) United States
(12) Patent Application Publication
(10) Pub. No.: US 2018/0000661 A1
(43) Pub. Date: Jan. 4, 2018

(54) ARRAY OF ABSORBENT ARTICLES

(71) Applicant: The Procter & Gamble Company, Cincinnati, OH (US)

(72) Inventors: Sarah Ann SANBORN, Cincinnati, OH (US); Theodore Cory Fites, Cincinnati, OH (US); Nguyen Huynh-Trang Le, Bad Soden Am Taunus (DE)

(21) Appl. No.: 15/639,093

(22) Filed: Jun. 30, 2017

Related U.S. Application Data
(60) Provisional application No. 62/356,562, filed on Jun. 30, 2016.

(51) Int. Cl. A61F 13/475; A61F 13/44; A61F 13/511; A61F 13/45

(52) U.S. Cl. CPC A61F 13/4756; A61F 13/51108; A61F 13/44; A61F 2013/4587

(57) ABSTRACT

Arrays of absorbent articles having signals or combinations of signals that help communicate functional features of the component article, including the presence and/or absence of channels.

### FIELD OF THE INVENTION

The present disclosure relates generally to personal hygiene absorbent articles, particularly an array of such articles and a system for facilitating consumers' identification and selection of the appropriate absorbent article configuration for a particular wearer.

### BACKGROUND OF THE INVENTION

Personal hygiene absorbent articles typically comprise an absorbent material disposed between a fluid-permeable skin-facing sheet (topsheet) and a fluid-impermeable garment-facing sheet (backsheet). Superabsorbent polymer (“SAP”) materials capable to absorb several times their own weight of urine are often used as absorbent material. All-cellulose fluff, SAP particles mixed with cellulose fluff, and cellulose-free absorbent cores (“airfelt-free” cores) are commercially available. The absorbent material may be enclosed in a discrete component called an absorbent core but it can also be more simply deposited directly between the topsheet and backsheet.

Such absorbent articles are typically offered in different sizes to accommodate the particular needs of different wearers. For example, baby care diapers may be segmented according to the weight of the wearer, each diaper size corresponding to a range of weight of the baby. The size of each articles increases with the weight ranges. The number of sizes offered and the corresponding weight ranges may vary between manufacturers and markets. Segmentation may also occur based on the baby's activity, where more active babies are provided with diapers configured to accommodate movement, with less activity configured to accommodate sleeping positions. Segmentation may also occur based on frequency and volume of urination, where increased frequency and higher volume babies are provided with diapers configured to absorb and retain more urine, and so on. Likewise, consumer preference for certain features may drive segmentation. Similar segmentation also exists in other categories of personal hygiene articles, such as feminine care and adult incontinence articles.

Recently, it has been proposed to provide absorbent structures that comprise SAP, optionally a cellulosic material, and at least a pair of substantially longitudinally extending absorbent material free zones that can form channels. Examples of such structures are disclosed in WO2012/170778 (Rosati et al., see also WO2012/170779, WO2012/170781 and WO2012/170808) and further in WO2014/093319, WO2014/093311 (both Arizti), WO2014/093310 (Ehrnsperger). The material free zone in these disclosures may be curved. Others disclose channels which are straight, and typically oriented in the longitudinal and/or transversal direction of the core, as in WO95/11652 (Tanzer) or WO2012/052,172A1 (Van Malderen).

US2015/0080837A1 (Rosati) discloses absorbent articles modified to communicate or signal certain functional elements of the absorbent core, e.g. the presence of channels in the core, such signaling or communicating being done, e.g. via exterior graphics and/or interior printed adhesives. WO2008/023291 (Cohen) discloses providing an array of disposable articles comprising different leg opening positions to accommodate children at different stages of development having various predominant leg positions. U.S. Pat. No. 7,582,075 (Betts) discloses the use of visual and narrative indicia on the packaging of disposable absorbent articles to aid a consumer in selecting the appropriate article for a particular wearer. However, it is not known to provide an array of absorbent articles comprising channels, where the absorbent articles making up the various components of the array indicate their place in the array using signaling or communicating characteristics which are embodied in the absorbent articles themselves.

### SUMMARY OF THE INVENTION

The present invention relates to arrays of absorbent articles having signals or combinations of signals that help communicate functional features of the component article, including the presence and/or absence of channels.

### BRIEF DESCRIPTION OF THE DRAWINGS

FIG. 1 is a schematic plan view of an exemplary absorbent article in the form of a taped diaper, with inner layers partially revealed, and comprising an absorbent core with a pair of curved channel-forming areas in accordance with a nonlimiting embodiment of the present invention;

FIG. 2 is a schematic plan view of the absorbent core in accordance with a nonlimiting embodiment of the present invention;

FIG. 3a is a schematic cross-section of the core of FIG. 2 taken along line 3-3;

FIG. 3b is the same transversal cross-section of the core of FIG. 3a after core has absorbed a fluid and swollen;

FIG. 4 is a schematic plan view of a close-up section of the central part of one channel-forming area in accordance with a nonlimiting embodiment of the present invention;

FIG. 5 is a schematic elevation view of an array in accordance with a nonlimiting embodiment of the present invention;

FIGS. 5A, 5B, 5C, and 5D are schematic plan views of exemplary indicia for component absorbent articles of an array in accordance with a nonlimiting embodiment of the present invention;

FIGS. 6A, 6B, and 6C, are schematic plan views of exemplary indicia for component absorbent articles of an array in accordance with a nonlimiting embodiment of the present invention;

FIGS. 7A, and 7B are schematic plan views of exemplary indicia for component absorbent articles of an array in accordance with a nonlimiting embodiment of the present invention; and

FIG. 8 is a schematic plan view of exemplary indicia for component articles of an array in accordance with a nonlimiting embodiment of the present invention, depicting indicia visible from the garment facing side and the body facing side.

### DETAILED DESCRIPTION OF THE INVENTION

### General Description of an Absorbent Article

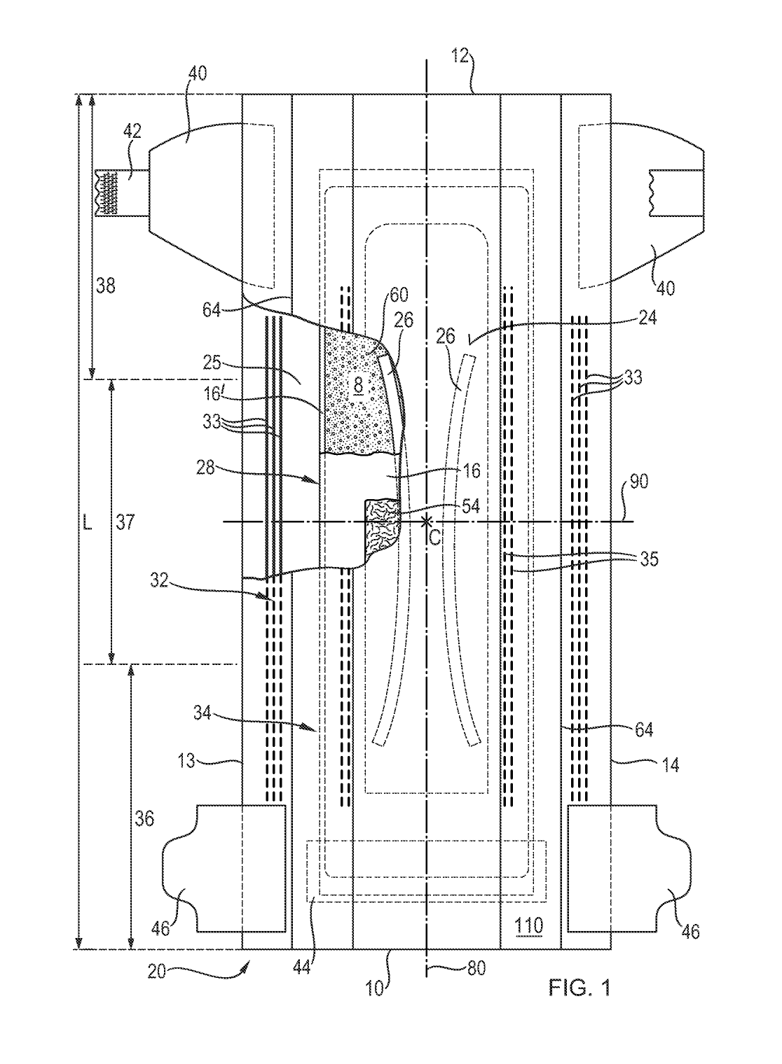

The invention relates to personal hygiene absorbent articles of the type used to absorb body exudates, e.g. urine, feces, and/or menses. The articles include baby and toddler diapers (including training pants), feminine sanitary pads and adult incontinence articles. An exemplary absorbent article according to the invention in the form of a baby taped diaper 20 is represented in FIG. 1. FIG. 1 is a top plan view of the exemplary diaper 20, in a flat-out state, with portions of the structure being cut-away to more clearly show the construction of the diaper. This diaper 20 is shown for illustration purpose only. The absorbent article can also be for example a pant-type article with pre-formed side seams. Unless otherwise indicated, dimensions and areas disclosed herein apply to the article in this flat-out configuration. If some part of the article is under tension due to elasticized components, the article may be typically flattened using clamps along the periphery of the article and/or a sticky surface, so that the topsheet and backsheet can be pulled taut so as to be substantially flat. Closed articles such as training pant may be cut open along the side seams to apply them on a flat surface.

The absorbent article 20 comprises a front edge 10, a back edge 12, and two longitudinally extending side (lateral) edges 13, 14 joining the front edge and the back edge. The front edge 10 is the edge of the article which is intended to be placed towards the front of the user when worn, and the back edge 12 is the opposite edge. The absorbent article is notionally divided by a longitudinal axis 80 extending from the front edge to the back edge of the article and dividing the article in two substantially symmetrical halves relative to this axis, when viewing the article from the wearer facing side in a flat out configuration, as exemplarily shown in FIG. 1. This axis 80 may typically be coincident with the longitudinal axis 80′ of the absorbent core. The article has a length L as measured along the axis 80 from the back edge to the front edge. The absorbent article 20 can also be notionally divided by a transversal axis 90 into a front region and a back region of equal length measured on the longitudinal axis, when the article is in such a flat state. This article's transversal axis 90 is perpendicular to the longitudinal axis 80 and placed at half the length of the article. The intersection of the longitudinal axis and transversal axis are referred herein as the Crotch Point “C”.

The absorbent article is further notionally divided in a front region, 36, a back region 38 and a crotch-region 37 in between. The front region 36 is defined as the region of the article extending from the front edge 10 and having a length of a third of L along the longitudinal axis 80. The back region 38 is defined as the region of article extending from the back edge 12 of the article and having a length of one third of L along the longitudinal axis 80. The crotch region 37 is the intermediate region between the front and back regions, and also having a length of a third of L along the longitudinal axis 80.

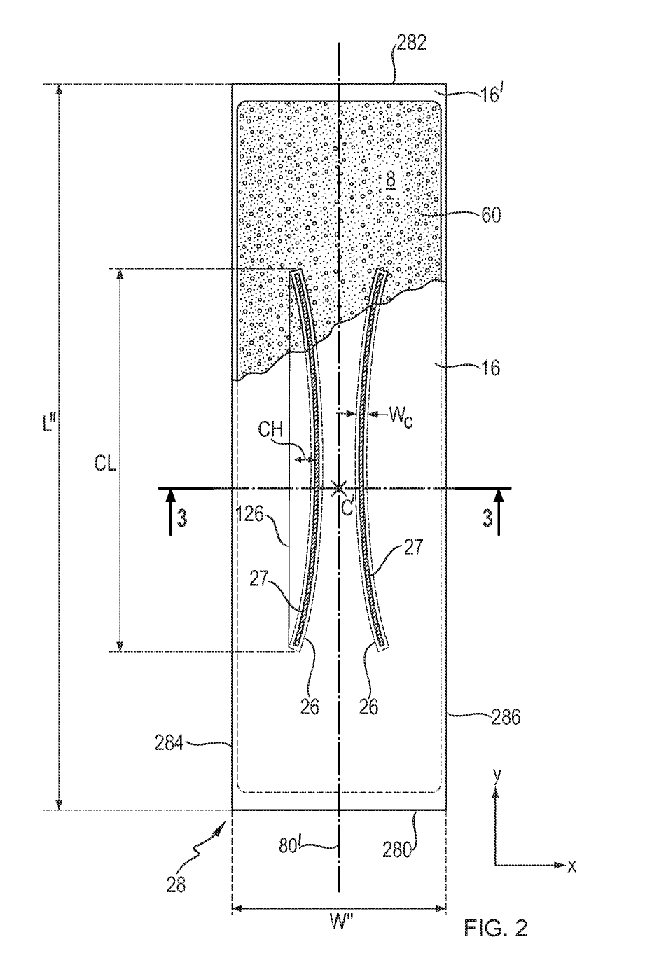

The absorbent article 20 comprises a wearer-facing side 110, which may be principally formed by a liquid permeable topsheet 24, a garment-facing surface 120 which may be formed by a liquid impermeable backsheet 25, and an absorbent core 28 between the topsheet 24 and the backsheet 25. The absorbent core 28 is shown in isolation in FIG. 2. The topsheet 24, the backsheet 25, the absorbent core 28 and the other article components may be assembled in a variety of well-known configurations, in particular by gluing and/or heat embossing. Exemplary diaper assemblies are for example generally described in U.S. Pat. No. 3,860,003, U.S. Pat. No. 5,221,274, U.S. Pat. No. 5,554,145, U.S. Pat. No. 5,569,234, U.S. Pat. No. 5,580,411, and U.S. Pat. No. 6,004,306. The absorbent article is preferably thin, for example with a caliper of from 2.0 mm to 8.0 mm, in particular from 3.0 mm to 6.0 mm, at the crotch point as measured using the Absorbent Article Caliper Test described below.

The article 20 may also comprise further components such as an acquisition layer and/or a distribution layer (collectively referred to as acquisition-distribution system “ADS”, designated as 54), elasticized gasketing cuffs 32 and upstanding barrier leg cuffs 34. The ADS may be profiled and/or curved towards the back edge of the article, for example as disclosed in WO2014/093323. FIG. 1 also shows other typical taped diaper components such as a fastening system comprising fastening tabs 42 attached towards the back edge 12 of the article and cooperating with a landing zone 44 towards the front edge 10 of the article. These fastening features are typically absent from pant-type articles which have a pre-formed side seam. The absorbent article may also comprise other typical components, which are not represented in the Figures, such as a back elastic waist feature, a front elastic waist feature, transverse barrier element, a wetness indicator that changes appearance when contacted with urine, a lotion application, etc. These components are well-known in the art and will not be further discussed herein. Reference is made to WO2014/093310 where several examples of these components are disclosed in more details.

Typically, adjacent layers will be joined together using conventional bonding method such as adhesive coating via slot coating, spiral gluing, or spraying on the whole or part of the surface of the layer, or thermo-bonding, or pressure bonding or combinations thereof. Most of the bonding between components is for clarity and readability not represented in the Figure. Bonding between the layers of the article should be considered to be present unless specifically excluded. Adhesives may be typically used to improve the adhesion of the different layers, for example between the backsheet and the core wrap a disclosed in WO2012/170341A1. The adhesives used may be any standard hotmelt glue as known in the art. The absorbent core 28 and the channel-forming areas 26 will now be further described.

As used herein, the term “absorbent core” refers to an individual component of the absorbent article and which comprises an absorbent material enclosed in a core wrap. The absorbent core as defined herein does not include the acquisition-distribution layer or multilayer system if present. The core wrap is typically formed by one or two layers of nonwoven or tissue materials, but it is not excluded that for some articles of simple construction the core wrap may be partially or entirely formed by the topsheet and/or the backsheet. The absorbent core is typically the component of an absorbent article that has the most absorbent capacity of all the components of the absorbent article and which comprises all, or at least the majority of, superabsorbent polymer (SAP). The core may consist essentially of, or consist of, the core wrap, the absorbent material and optionally adhesives. The terms “absorbent core” and “core” are herein used interchangeably.

The absorbent cores of the invention are substantially planar. By substantially planar, it is meant that the absorbent core can be laid flat on a planar surface. The absorbent cores may also be typically thin and conformable, so that they can also be laid on a curved surface for example a drum during the making process, or stored and handled as a continuous roll of stock material before being converted into an absorbent article.

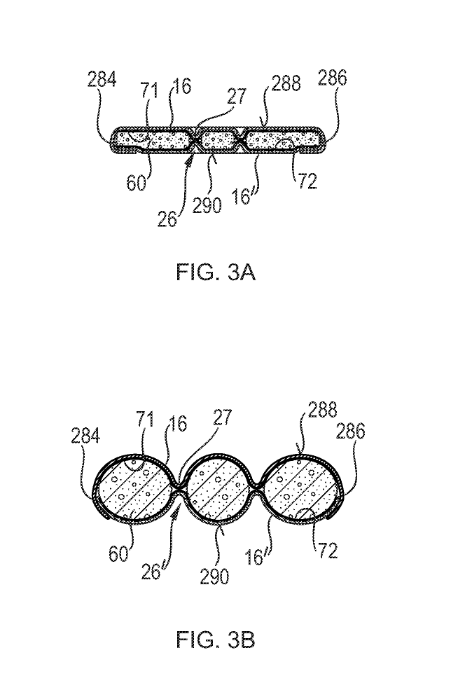

For ease of discussion, the exemplarily absorbent core of FIGS. 2-3 is represented in a flat state. The absorbent core is relatively thin relative to its other dimensions in the transversal direction (x) and the longitudinal direction (y). These directions correspond to the transversal and longitudinal direction of the article respectively. Unless otherwise indicated, dimensions and areas disclosed herein apply to the core in this flat-out configuration. The same applies to an absorbent article, as exemplarily represented in FIG. 1 as a taped diaper, in which the core is integrated. The absorbent cores and articles of the invention are discussed with reference to the Figures and the numerals referred to in these Figures; however these are not intended to limit the scope of the claims unless specifically indicated.

The absorbent cores 28 illustrated comprise a front edge 280, a back edge 282 and two longitudinal side edges 284, 286 joining the front edge and the back edge. The front edge of the core is the edge intended to be placed towards the front edge of the absorbent article in which the core is or will be integrated. Typically the front and back edges 280, 282 of the core may be shorter than the longitudinal side edges 284, 286 of the core. The absorbent core also comprises a top side 288 and a bottom side 290. The top side of the core is placed or intended to be placed towards the wearer-facing side (topsheet 24) of the article and the bottom side is the side placed or intended to be placed towards the garment-facing side (backsheet 25) in the finished article. The top side of the core wrap may be more hydrophilic than the bottom side, for example after treatment with a wetting agent.

The absorbent core may be notionally divided by a longitudinal axis 80′ parallel to the longitudinal direction y and extending from the front edge 280 to the back edge 282 and dividing the core in two substantially symmetrical halves relative to this axis, when viewing the core in the plane formed by the longitudinal and transversal direction (x, y). The length L″ of the core is measured from the front edge 280 in direction of the back edge 282 along the longitudinal axis 80′, including the region of the core wrap which does not enclose the absorbent material, in particular at the front and back end seals when present. The crotch point of the core C′ is the point that is aligned vertically with the crotch point C of the absorbent article and normally also placed on the longitudinal axis 80′ of the absorbent core.

The width W″ of the core is the maximum dimension of the core wrap measured along the transversal direction x, which is perpendicular to y. The outline of the absorbent core defined by the core wrap can typically be generally rectangular. The width W″ and length L″ of the core may vary depending on the intended usage. The core wrap may also be shaped, with a width at the crotch point C′ narrower than at the front and/or back of the core wrap. For baby care applications such as diapers and infant training pants for example, the width of the core may typically ranges from 4 cm to 22 cm and the length from 10 cm to 62 cm depending on the size and capacity desired. The ratio length to width (L″/W″) may for example range from 2 to 10. Adult incontinence articles may typically be longer and larger than baby articles and have even higher dimensions.

The absorbent core comprises an absorbent material 60 encompassed within the core wrap. The absorbent material may comprise a high proportion of superabsorbent polymer (herein abbreviated as “SAP”). The term “superabsorbent polymer” refers herein to absorbent materials, which may be cross-linked polymeric materials, and that can absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 25 to 40 g/g. The SAP content may represent at least 80% and up to 100% by weight of the absorbent material contained in the core wrap. The SAP may in particular be in particulate forms (SAP particles) but other forms are also possible, such as absorbent foam or fibers. Further detailed examples of absorbent material, in particular SAP are disclosed in WO2014/093310 (Ehrnsperger). In particular, the absorbent material may comprise or consist of SAP particles that require a time to reach an uptake of 20 g/g (T20) of less than 240 s as measured according to the K(t) test method described in WO2012174026.

The absorbent material may in particular be substantially free of cellulose fibers, meaning it comprises at least less than 20% by weight of cellulose fibers relative to the total weight of absorbent material, in particular less than 10%, or less than 5% and down to 0% by weight of cellulose fibers. The absorbent core may thus be relatively thin, in particular thinner than conventional cores comprising cellulosic fibers. In particular, the caliper of the core (before use) as measured at the crotch point (C′) or at any other points of the surface of the core according to the Core Caliper Test as described herein may be from 0.25 mm to 5.0 mm, in particular from 0.5 mm to 4.0 mm.

The absorbent material 60 forms an absorbent material deposition area 8 within the core wrap. The deposition area 8 may comprise a continuous layer of absorbent material in the plane of the core, as shown in FIG. 2. Alternatively, the deposition area may comprise discrete or joined land areas which comprise absorbent material and material free junction-areas in-between. The basis weight (amount deposited per unit of surface) of the absorbent material may also be varied to create a macroscopic profiled distribution of absorbent material in the longitudinal direction (y) and/or the transversal direction (x). Typically the absorbent material of the core may be advantageously distributed in somewhat lower amount towards the back portion 38 of the core as more absorbency is typically required towards the crotch region and front region. Other absorbent material distributions are however possible, for example a homogenous distribution across the absorbent material area 8 may be easier to make.

The absorbent material deposition area 8 may be generally rectangular, as illustrated in FIG. 2, but shaped absorbent material deposition area are also desirable. A typical shaped deposition area tapers in the crotch region, which has at least a point where its width is smaller than the width of the absorbent deposition material area at the front and/or back regions, in particular forming dog-bone or sand-hour shaped area absorbent material area. Further detailed examples of absorbent material distribution that can be used herein are disclosed in WO2014/093310 (Ehrnsperger).

Various absorbent core designs comprising high amount of SAP have been proposed in the past. The processes used can allow relatively precise deposition of SAP at relatively high speed, and which may be used to make the absorbent cores of the invention, see for example in U.S. Pat. No. 5,599,335 (Goldman), EP1,447,066 (Busam), WO95/11652 (Tanzer), US2008/0312617 (Hundorf), WO2012/052172 (Van Malderen) and in particular WO2012/170778 (Rosati et al., see also WO2012/170779, WO2012/170781 and WO2012/170808).

The absorbent material may be deposited for example using a SAP printing technology as disclosed in US2006/024433 (Blessing), US2008/0312617 and US2010/0051166A1 (both to Hundorf et al.). This technique uses a transfer device such as a printing roll to deposit SAP particles onto a substrate disposed on a grid of a support (e.g. a lay-on drum). The grid may include a plurality of cross bars extending substantially parallel to and spaced from one another so as to form ribs extending between the cross-bars. The SAP is deposited inside these ribs. This technology allows high-speed and precise deposition of SAP on a substrate in particular to provide areas substantially free of absorbent material surrounded by absorbent material through which the core wrap can be bonded to itself to form channel-forming areas. The areas substantially free of absorbent material can be formed for example by providing an area on the support where no SAP is applied and then bonding both sides of the core wrap to each other through these areas free of absorbent material. One way to achieve this is exemplary disclosed in US2012/0312491 (Jackels).

The core wrap may be formed by any substrate materials suitable for receiving and containing the absorbent material. Typical substrate materials used are in particular nonwovens, paper, tissues, films, wovens, or laminate of any of these. The core wrap may in particular be formed by a nonwoven web, such as a carded nonwoven, spunbond nonwoven (“S”) or meltblown nonwoven (“M”), and laminates of any of these. For example spunmelt polypropylene nonwovens are suitable, in particular those having a laminate web SMS, or SMMS, or SSMMS, structure, and having a basis weight range of about 5 gsm to 15 gsm. Suitable materials are for example disclosed in U.S. Pat. No. 7,744,576, US2011/0268932A1, US2011/0319848A1 and US2011/0250413A1. Nonwoven materials provided from synthetic fibers may be used, such as PE, PET and in particular PP.

The core wrap may, as shown in the cross-sectional view of FIG. 3, comprise a first substrate 16 forming the top side 288 of the core and a second substrate 16′ essentially forming the bottom side 290 of the core. Alternatively, it is also known in the art to make a core wrap out of a single substrate, or to use the backsheet or topsheet as substrate to directly, partially or completely form the core wrap. When two substrates are used, the core wrap may have a C-wrap along each longitudinal side edges 284, 286 of the core. The core wrap is not considered as absorbent material for the purpose of calculating the percentage of SAP in the absorbent core. Examples of core wrap construction are further detailed in WO2014/093310.

The absorbent core may comprise one or more layers of glue to help immobilizing the absorbent material. The absorbent core 28 may comprise at least one auxiliary glue 71, 72 layer applied on the inner surface of the top side and/or the bottom side of the core wrap. The auxiliary glue may be applied directly over the substrate on which the absorbent material is subsequently deposited, thus at least partially immobilizing the absorbent material on the substrate. The auxiliary glue may also at least partially form the core wrap bond 27 of the channel-forming areas 26. The auxiliary glue may also be useful to improve the adhesion of a fibrous thermoplastic material, when present, to the substrate. The auxiliary glue can be applied by any adhesive applicator known in the field, in particular bead, slot or spray nozzles. For example, the auxiliary glue can be applied using a slot coating process as a pattern comprising a plurality of spaced-apart slots which may each extend in the longitudinal direction. The slots may for example have a width of from 0.5 mm to 3 mm, and/or have a lateral spacing there-between of from 0.5 mm to 4 mm.

The absorbent core 28 may also comprise a fibrous thermoplastic adhesive material (not shown), also known as microfiber glue, to help immobilizing the absorbent material 60 within the core wrap. The fibrous thermoplastic adhesive material may be applied, typically by spraying, over an absorbent material that has been discontinuously deposited on a substrate during the core making process, thus forming land and junction areas as indicated above. The fibrous thermoplastic adhesive material contacts the absorbent material and the substrate layer in the absorbent material free junction areas. This imparts an essentially three-dimensional net-like structure to the fibrous layer of thermoplastic adhesive material, which in itself is essentially a two-dimensional structure of relatively small thickness, as compared to the dimension in length and width directions. Thereby, the fibrous thermoplastic adhesive material may provide cavities to cover the absorbent material, and thereby immobilizes this absorbent material. A dual layer core can thus be constructed wherein the land areas of one layer correspond to the material-free junction areas of the other layer and vice versa, resulting in continuous dual absorbent layer.

More details about these glues and how they can be applied in a SAP printing process is further generally disclosed in US2006/024433 (Blessing), US2008/0312617 and US2010/051166A1 (both to Hundorf) and US2014/027066A1. The absorbent cores may advantageously provide a sufficient immobilization of the absorbent material in dry and wet state. The absorbent core advantageously achieves an SAP loss of no more than about 70%, 60%, 50%, 40%, 30%, 20%, or 10% according to the Wet Immobilization Test described in US2010/051166A1.

### Channel-Forming Areas 26

One or more of the absorbent articles forming a component of an array may further comprise at least a pair of channel-forming areas 26 which will now discussed in more details. The channel-forming areas 26 may each comprise an absorbent material free area within which a core wrap bond 27 is present. The channel-forming areas may typically be not visible in the article before use because of the opacity of the other layers of the article, in particular when the absorbent core is substantially free of cellulose fibers and thus only a few mm thin. The thickness of the core 28 when dry, as represented in FIG. 3, is exaggerated to clearly show the channel-forming areas 26 and core wrap bond 27.

The channel-forming areas at least initially do not substantially swell as the absorbent core absorbs urine or another fluid, while the absorbent material adjacent the channel-forming areas can swell considerably. The channel-forming areas thus forms three-dimensional channels when the absorbent material adjacent the channel-forming areas absorbs a fluid and swells. As the core absorbs more liquid, the depressions within the absorbent core formed by the core wrap bond 27 between the two substrates become deeper and apparent to the eye and the touch. With sufficiently strong core wrap bond, and/or a relatively extensible substrate material, the channels can remain permanent until complete saturation of the absorbent material. On the other hand, the core wrap bonds 27 may be designed to at least partially open or break when the core is close to saturation so as not to unduly restrict the swelling of the absorbent material when the core is substantially loaded.

The channel-forming areas may be formed in different manners. In particular, channel-forming areas may be areas of the core that are substantially free of absorbent material and through which the top side of the core wrap is bonded to the bottom side of the core wrap. The core wrap bond 27 between the top side and bottom side of the core wrap in these areas may be at least partially formed by auxiliary glue 71, 72 and/or microfiber glue (not shown) applied directly to the inner surface of at least one of the substrate. This is exemplarily disclosed in WO2012/170778 for example. However it is also known to form bonds using other bonding means such as ultra-sonic bonding, pressure bonding or heat bonding. The channel-forming areas can also possibly not comprise such core wrap bonds, but the resulting channels may then be less resistant to compression and the absorbent material may fill the channel areas relatively quickly when the article is worn. The channel-forming areas may also be formed by a core wrap bond with minimal or no surrounding absorbent material free areas.

The channel-forming areas 26 are pair-wise symmetrically disposed on opposite side of the longitudinal axis of the article 80 and are concave towards the longitudinal axis 80 of the article. By “concave” towards the longitudinal axis” it is meant that the channels have a point or area which is closest to the longitudinal axis (typically in the crotch region of the article) and that the channels generally diverge from this closest point or area as the channels extend towards the front and back of the article. The smallest distance or gap between the pair of areas may be for example at least 5 mm, or at least 10 mm, or at least 16 mm and may for example be up to 40 mm.

The channels may be advantageously curvilinear. The curve may have a substantially constant radius of curvature along the length of the curved portion or this radius may vary. It also not excluded that at least portion of the channels may be straight and/or comprise a plurality of straight segments. For examples, the channel-forming areas could be approximated by two lines meeting at the point closest to the longitudinal axis and forming together a flat V. The channel-forming areas may also comprise three or more segments.

Although not represented in the Figures, it is not excluded that a portion of the channel-forming areas may not be concave towards the longitudinal axis, for example that a portion of the channels at the extremities toward the front and/or back edge of the core may be parallel to the longitudinal axis or even convex (converging towards the longitudinal axis). The absorbent core may comprise further channel-forming areas in addition to the curved pairs, in particular channel-forming areas that may not be curved or that may be placed in the front region of the article only as disclosed in WO2012/170779.

The channel-forming areas 26 may be substantially free of absorbent material, so that the bond 27 between the top side 288 and bottom side 290 of the core wrap can be easily formed within these areas substantially free of absorbent material, for example by gluing and/or pressure bonding these two surfaces together. By “substantially free of absorbent material” it is meant that there can be practically no absorbent material in these areas. Minimal amount such as involuntary contaminations with absorbent material particles that may occur during the making process are disregarded. Within the channel-forming areas, the top side 288 of the core wrap is attached to the bottom side 290 by a core wrap bond 27 as illustrated in FIGS. 2-3. The portion of the channel-forming areas immediately adjacent the bond 27 may be substantially free of absorbent material. The channel-forming areas as a whole including this absorbent material free area may have a width Wc. In this example, the width of the channel-forming area 26 which corresponds to an area substantially free of absorbent material may be of about 8 mm and the channel bond 27 may be of about 2 to 3 mm. The channel-forming areas may be substantially free of absorbent material along at least part of their length across a width Wc which is at least 2 mm, or at least 3 mm or at least 4 mm, up to for example 20 mm, or 16 mm or 12 mm. The width We of the areas substantially free of absorbent material may be constant through substantially its whole length or may vary along the length of the channel-forming areas.

The channel-forming areas 26 and bonds 27 are advantageously substantially surrounded by the absorbent material 60, when considering the plane of the core. In particular they may advantageously not extend to any of the edges of the absorbent material deposition area to reduce the risk of leakage. Typically, the smallest distance between a channel-forming area and the closest edge of the core wrap may be at least 5 mm or advantageously at least 10 mm.

When the absorbent material 60 swells upon absorbing a fluid, the core wrap bonds 27 remain at least initially attached in the channel-forming areas 26. The absorbent material 60 having swollen in the rest of the core, the core wrap forms channels, i.e. elongated depressions, along the core wrap bond 27, as illustrated in FIG. 3b. These channels 26′ are three-dimensional and have been found to function as bending lines which drive the bending of the diaper to conform to the wearer's anatomy and therefore improve the fit of the diaper. The channels can also serve to distribute an insulting fluid along their length to a wider area of the core. They may provide a quicker fluid acquisition speed and a better utilization of the absorbent capacity of the core. The channels can also provide a deformation of an overlying layer such as a fibrous layer 54 and provide corresponding ditches in the overlying layer. The absorbent core may comprise other areas substantially free of absorbent material, such as the spaces between the absorbent material areas, but without a core wrap bond, these non-bonded areas will typically not form durable three-dimensional channels.

The core wrap bond 27 may also be designed to gradually open in a controlled manner when exposed to a large amount of fluid. The bonds may thus remain substantially intact at least during a first phase as the absorbent material absorbs a moderate quantity of fluid. In a second phase the core wrap bonds 27 in the channels can start opening to provide more space for the absorbent material to swell while keeping most of the benefits of the channels such as increased flexibility of the core in transversal direction and fluid management. In a third phase, corresponding to a very high saturation of the absorbent core, a more substantial part of the channel bonds can open to provide even more space for the swelling absorbent material to expand. The strength of core wrap bond 27 within the channels can be controlled for example by varying the amount and nature of the glue used for the attaching the two sides of the core wrap, the pressure used to make the core wrap bond and/or the distribution of the absorbent material, as more absorbent material will usually causes more swelling and will put more pressure on the bond. The extensibility of the material of the core wrap may also play a role.

The core wrap bond 27 may be continuous along each channel-forming area 26 but it may also be discontinuous (intermittent) such as formed by series of point bonds. Each channel-forming areas 26 is also advantageously continuous but it is not excluded that it may be comprised of discrete section separated by small gaps as long as a generally concave outline is formed by the discrete sections. An auxiliary glue 71, 72 when present may at least partially help forming the bond 27. Typically, some pressure can be applied on the substrates in the areas 26 so that the auxiliary glue better forms the bonds between the substrates. Of course it is not excluded that the core wrap bond 27 is made via other known attachment means, such as pressure bonding, ultrasonic bonding or heat bonding or combination thereof, in which case the width of the channel-forming areas may be even more reduced. If an auxiliary glue 71, 72 is applied on the inner surface of any of the substrates 16, 16′ as a series of longitudinally-oriented continuous slots, the width and frequency of these slots may advantageously be such that at least one slot of auxiliary glue is present at any level of the channel in the longitudinal direction. For example the slots may be 1 mm wide with a 1 mm distance between neighboring slots, and the absorbent material free areas forming the channel-forming areas have a width of about 8 mm. In this example, 4 slots of auxiliary glue will be present on average in each of the areas 26.

In general, the core wrap bonds 27 may have the same outline but be slightly smaller than the absorbent material free areas in which they are formed due to the tolerance required in some manufacturing process.

The channel-forming areas 26 are at least partially present in the crotch region 37 of each article; in particular they can be sufficiently elongated to extend through the whole of the crotch region and into the front region and/or the back region of the article. The channel-forming areas 26 may thus have a length CL which is at least 25% of the length L of the article, in particular at least 33% or even at least 40% of L, up to 90% or 80% of L.

The channel-forming areas 26 are further characterized herein by the ratio of their channel length (CL) to the channel height (CH). The channel length (CL) corresponds to the distance between the two extremities of the channel-forming areas, as illustrated in FIG. 2 by the line 126, including the absorbent-material free area if present. If the channel-forming areas have a measurable width Wc, the center of the channel-forming areas at the extremities is used to measure the channel length.

The straight line 126 joining these two extremities may be generally parallel to the longitudinal axis 80′ of the core (see FIG. 2) but it is not excluded that this line may have an angle relative to the longitudinal axis if one of the extremities is skewed transversally relative to the other extremity. Such an angle may for example be up to 25°, or up to 20° relative to the longitudinal axis 80 of the article or the longitudinal axis 80′ of the absorbent core.

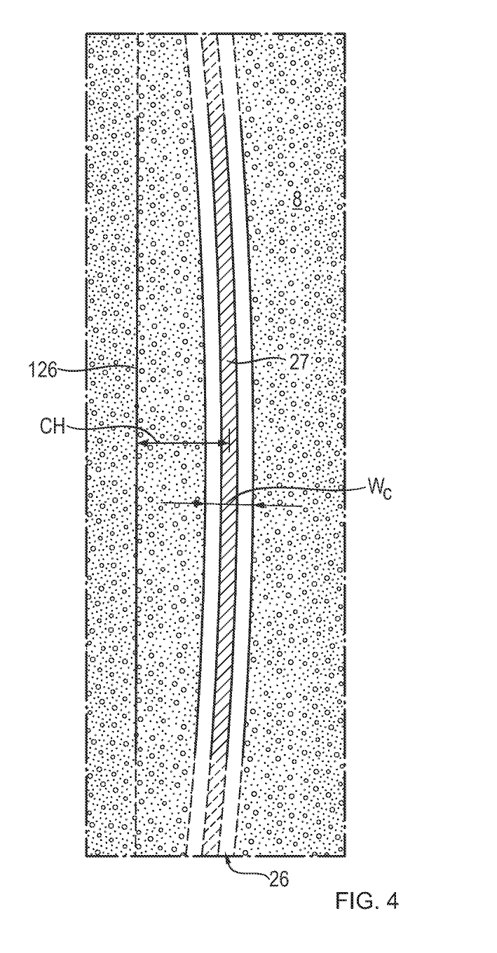

The channel height (CH) is the largest distance between the line 126 joining the two extremities of the channel-forming areas and the rest of the channel-forming area, as measured perpendicularly to the joining line 126. This is illustrated in FIG. 4, which is a close-up of the relevant part of FIG. 2 where the distance CH is measured. If the channel forming-areas 26 have a measurable width, as is illustrated, the channel height is measured by referring to the central portion of the channel-forming area 26, which may typically be the center of the bond 27, as shown on FIG. 4.

Additional layers, such as a layer of the ADS, may comprises channels, which may or may not coincide with channel-forming areas 26 that are present in the core.

Signals

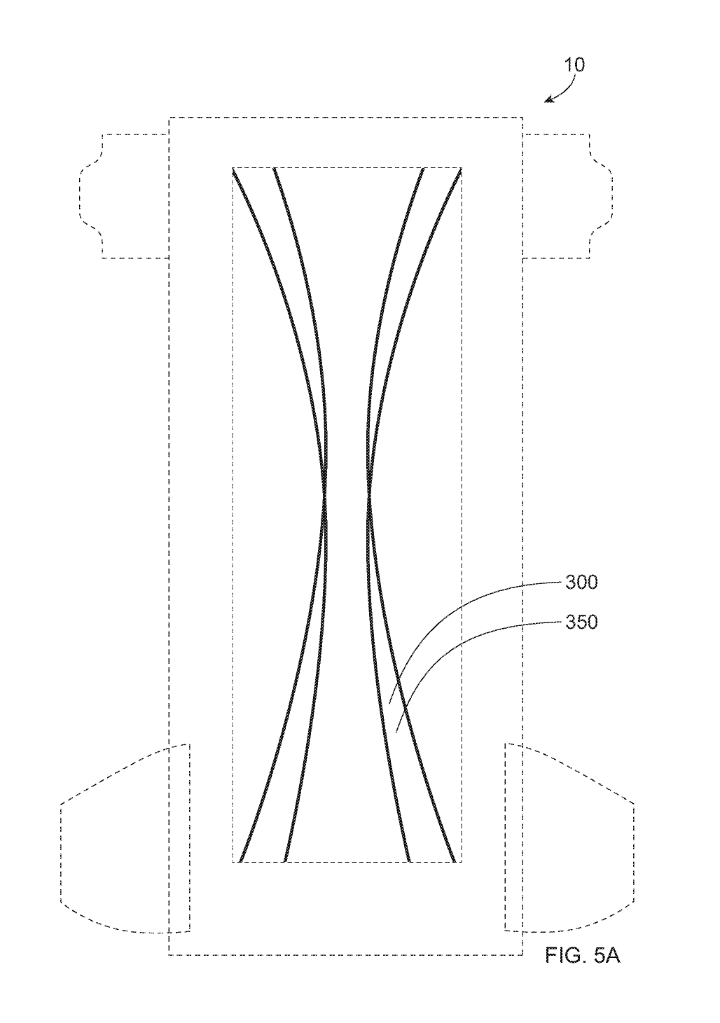

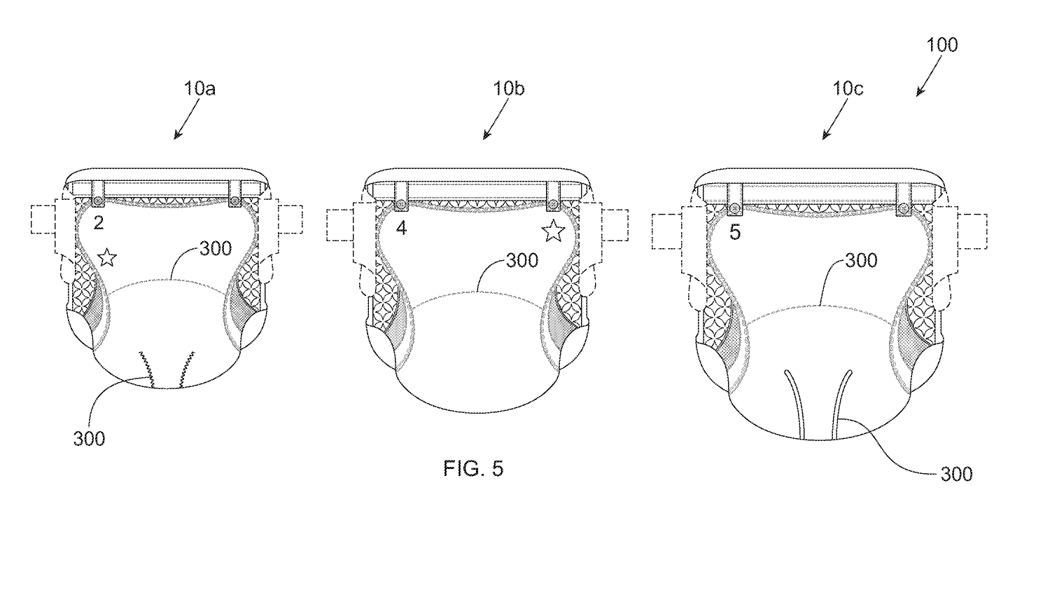

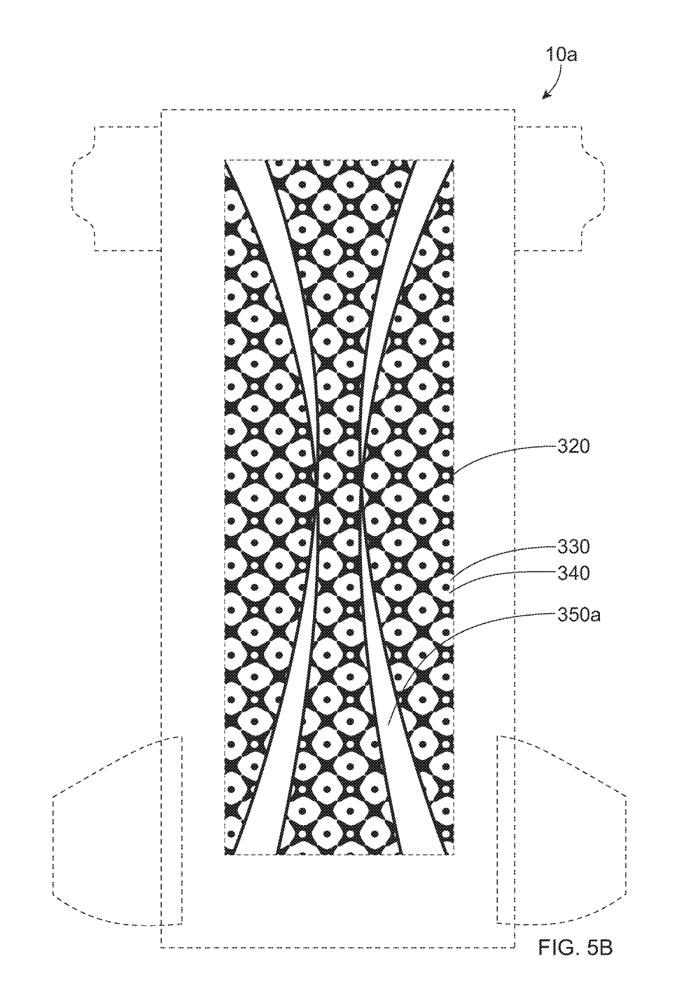

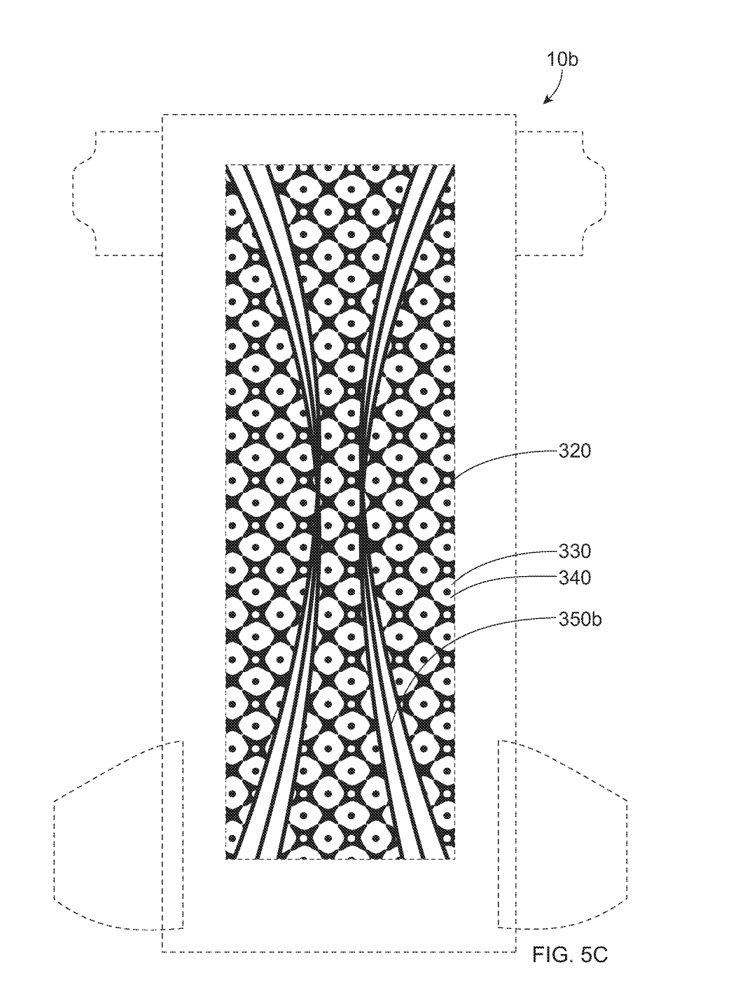

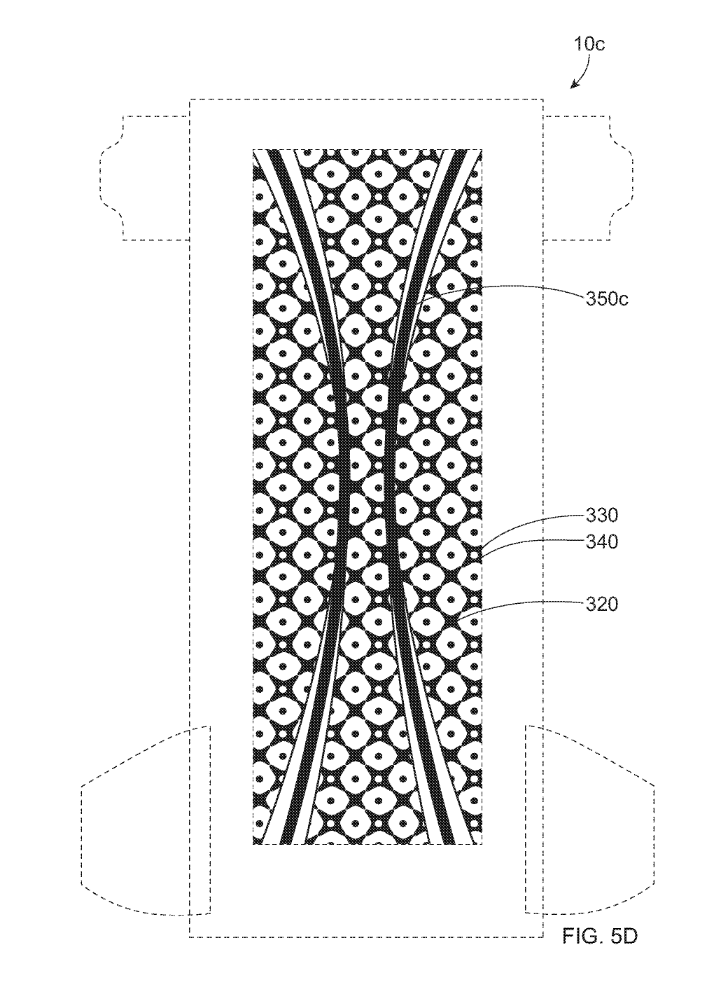

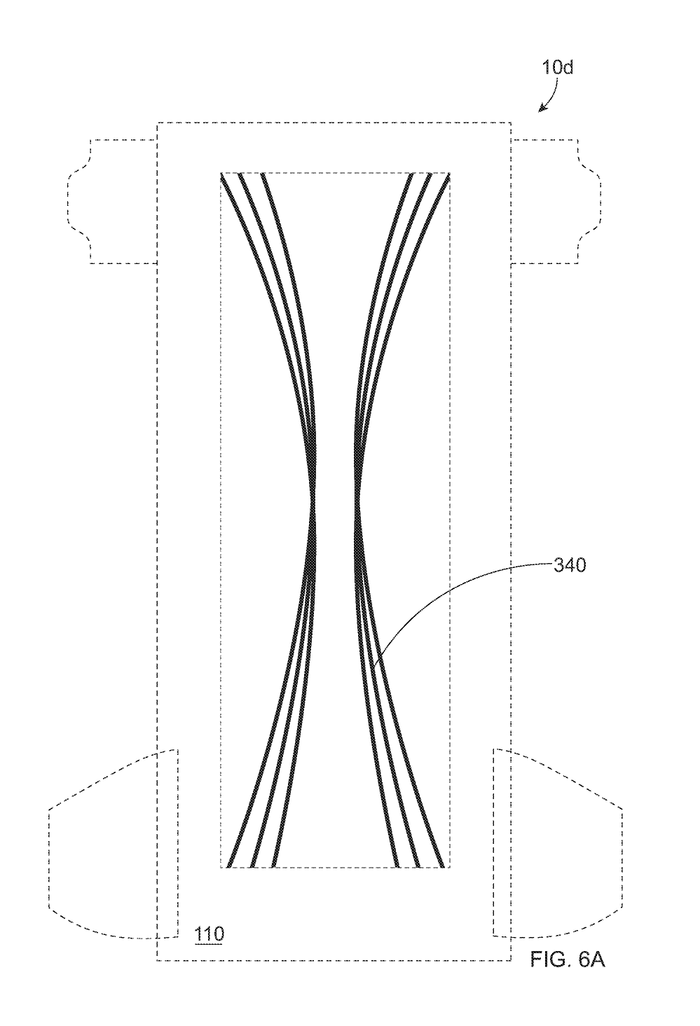

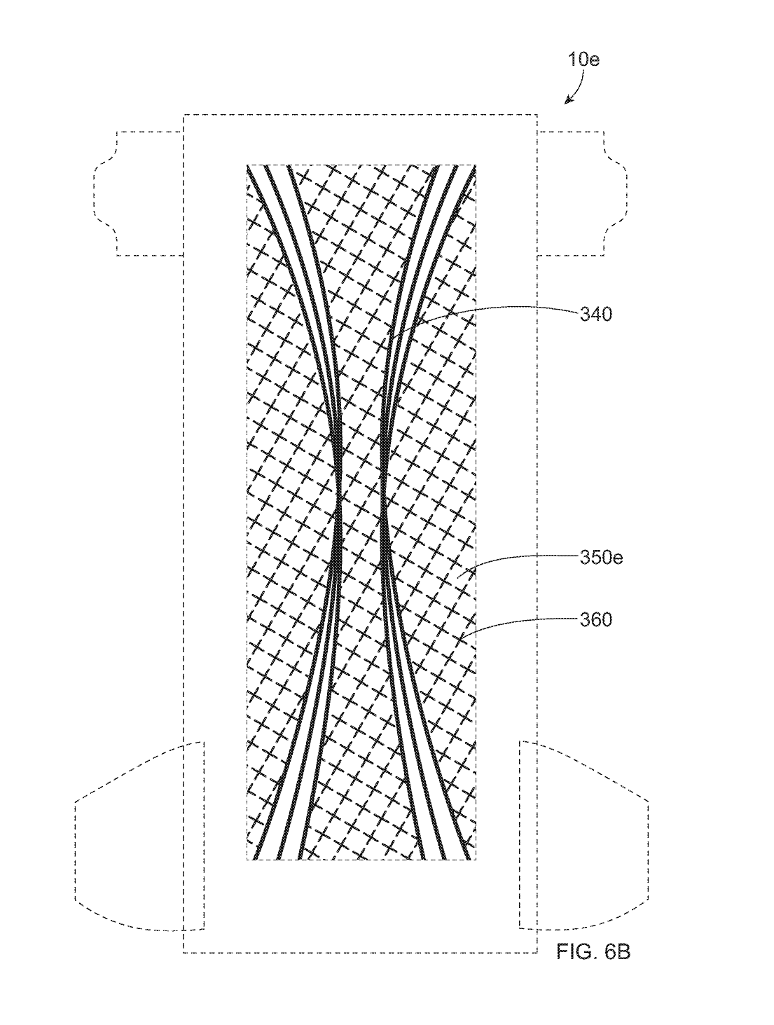

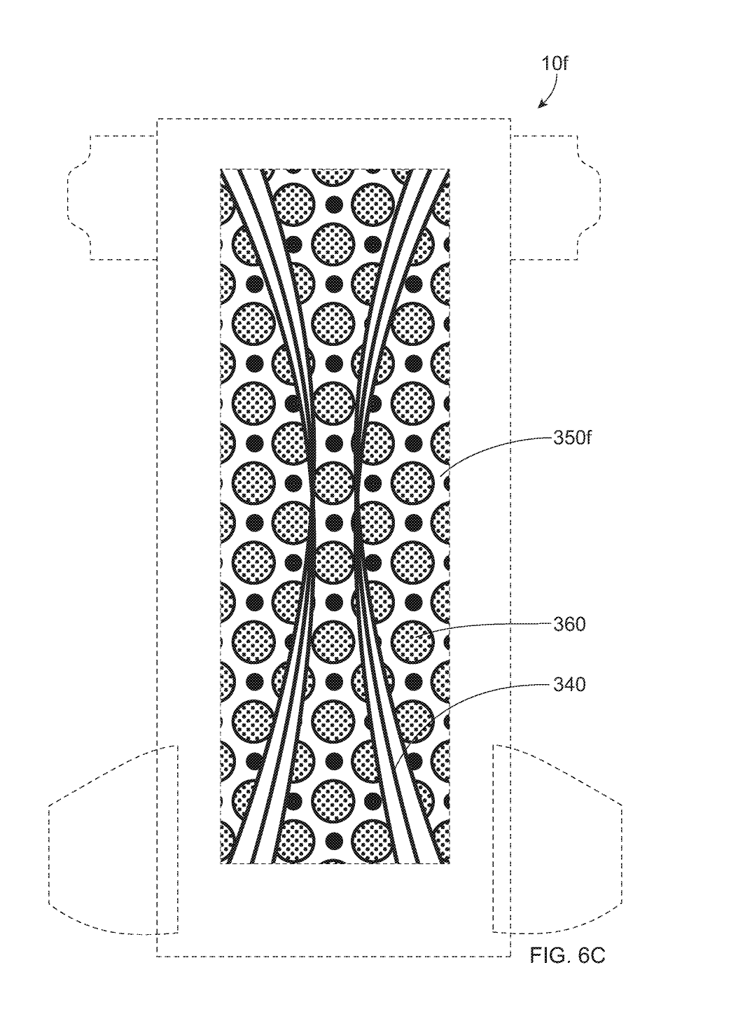

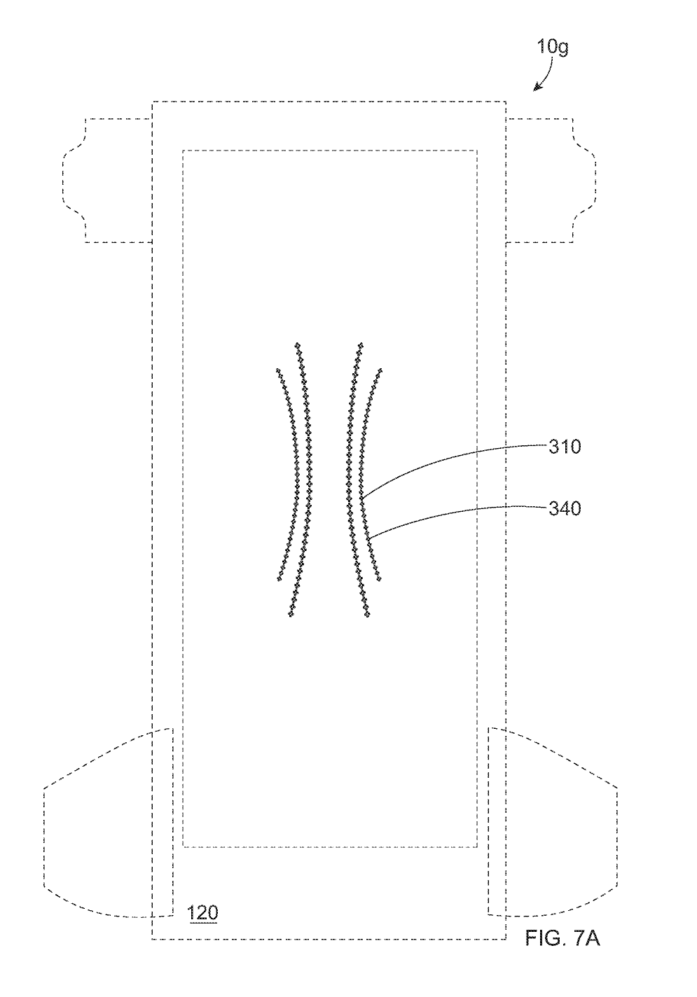

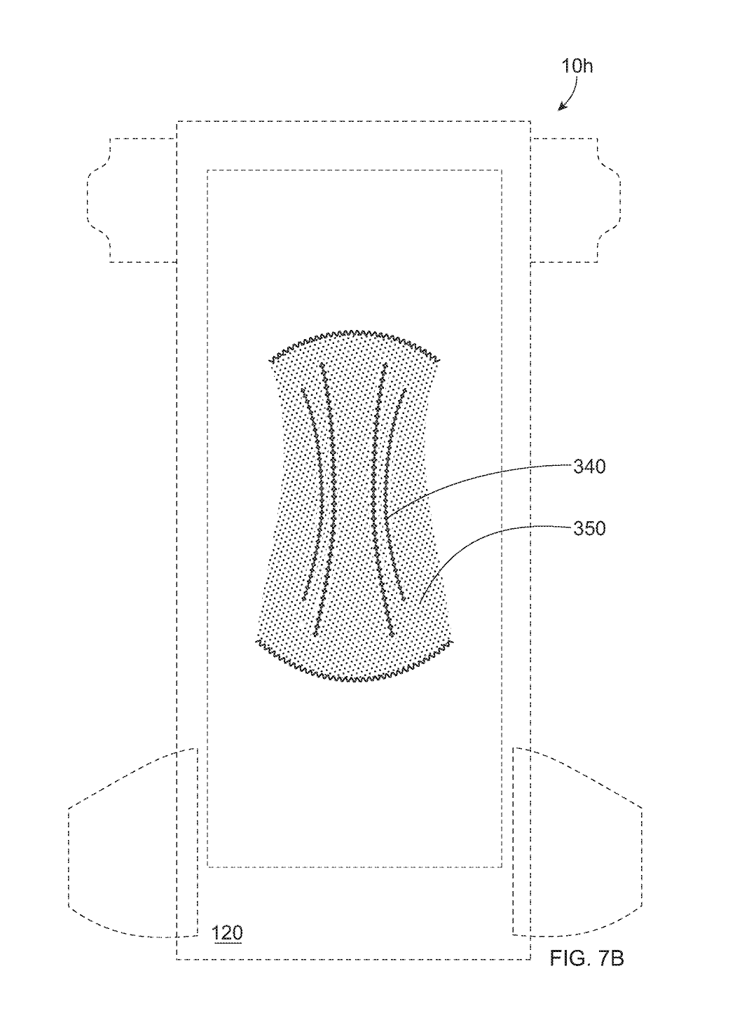

As shown for example, in FIGS. 5A-5D, absorbent articles of the present invention may comprise signals 300 that communicate to the consumer the functionality and benefits of the absorbent articles, including but not limited to channels, size, absorbency, softness, environmental friendliness, configuration for movement/activity of the wearer, water resistance and combinations thereof. Non-limiting examples of such signals may include printed layers 320 (including for example printed ink layers, printed adhesive layers, printed colored adhesive layers), backsheet graphics 310 (e.g., FIG. 7A), embossing 360 (e.g., FIG. 6B), changes in shape upon liquid insult (e.g., depressions in channel areas, color-changing agents to indicate wetness to the user) and combinations thereof. In some embodiments, the signal may be provided in a pattern 330. The term “pattern” as used herein means a decorative or distinctive design, not necessarily repeating or imitative, including but not limited to the following: marbled, check, mottled, veined, clustered, geometric, spotted, helical, swirl, arrayed, variegated, textured, spiral, cycle, contoured, laced, tessellated, starburst, lobed, lightning, blocks, textured, pleated, cupped, concave, convex, braided, tapered, and combinations thereof.

One type of signal 300 that may be used to communicate the existence and the benefits of a functional characteristic 400, such as channels (see e.g., FIGS. 3A-3B), is a printed layer 320 of ink, adhesive, or both, i.e. a colored adhesive layer. In some embodiments, an article with channels in the core may have a such a layer that is applied in a pattern that connotes the absorbency and other benefits of the channels, including the presence and location of the channels which may not otherwise be noticeable from the exterior of the absorbent article (whether on the garment-facing side 120 or the skin-facing side 110). Such layer may be applied to a substrate, such as a supporting layer, in some embodiments an acquisition layer 54, that may be positioned between a topsheet and the absorbent core with channels. It is contemplated that such a layer may be applied to, or be a part of, the skin-facing surface or the garment-facing surface of any one or more of the components of the absorbent article, including the topsheet, an acquisition layer, a distribution layer, a core wrap, a core, a backsheet (including laminates of film and nonwovens, where such layer may be applied to or be a part of any surface of the layers making up such laminate), and combinations thereof. In some embodiments, such layer may be visible through the topsheet (i.e., when viewing the topsheet) even if not applied directly to the topsheet. It should be understood that by signaling the presence of channels, a signal also indicates the presence of channel-forming areas and vice versa.

In some embodiments, a fluid, such as ink, an adhesive, or a blend thereof, may be applied or printed onto an advancing substrate. The fluid application apparatus may include a slot die applicator and a substrate carrier. The slot die applicator may include a slot opening, a first lip, and a second lip, the slot opening located between the first lip and the second lip. And the substrate carrier may include one or more pattern elements and may be adapted to advance the substrate past the slot die applicator as the slot die applicator discharges fluid onto the substrate. In operation, when the first surface of the substrate is disposed on the substrate carrier, the substrate carrier advances the second surface of the substrate past the slot opening of the slot die applicator. In turn, the substrate is intermittently compressed between the slot die applicator and the pattern surface of the pattern element. As the substrate is intermittently compressed, fluid discharged from the slot die applicator is applied onto the second surface of the advancing substrate in an area having a shape that is substantially the same as a shape defined by the pattern surface. In some embodiments, the fluid may be a different color than the substrate. The fluid may comprise pigments or dyes. Other methods and apparatuses involved for the application of adhesives in pre-determined patterns to an advancing substrate are disclosed in U.S. Pat. No. 8,186,296. In some embodiments, the fluid printed on the substrate may be an ink without an adhesive. The printing (ink or an adhesive with ink) may be on the topsheet itself, and where ink is the substance being printed, the printing may be performed using digital printing.

Examples of signals embodying indicia that approximates the shape and contour of channels, indicia provides a “background” pattern, or both, may be seen in FIGS. 5A through 9E. In these figures, the curved lines approximating the channels and the pattern may signal to a consumer the presence of channels, and/or one or more benefits of the channels. For example, the channels may act as a conduit, distributing fluid to a more comfortable place, while the signal (which may be in the form of a printed adhesive layer, visible to the caregiver through the topsheet) may signal or communicate the fluid distribution and comfort that the article can provide. While illustrated as continuous smooth lines, it is also within the scope of the invention that lines within a signal may be discontinuous, zig zag, comprised of shapes as in FIG. 7A, or any other suitable configuration.

In some embodiments, the absorbent article may comprise graphics 310 printed onto the backsheet. As the absorbent core is loaded with fluid, the absorbent material will expand in particular towards the backsheet, while the channels which do not comprise absorbent material will not expand. This difference may be used, as the channels will become more perceptible through the backsheet as fluid is absorbed as they may form depressions. These depressions will become more pronounced as the absorbent article absorbs the fluid.

The depth of these depressions formed by the channels on the backsheet side will be proportional to the amount of fluid absorbed, and the inventors have found that that the visual appearance to the caregiver may be improved by providing a backsheet printed signal 310 which matches or at least indicates the region of the channels to the caregiver. Thus the backsheet printing may comprise a line or a curve which substantially matches the shape and/or position of the channels.

Backsheet graphics, for example, as shown in FIGS. 7A-7B, may similarly signal to the caregiver the attributes of the channels. In some embodiments, the only visual signal of the channels may be the backsheet printing. In some embodiments, the backsheet printing may be curves, lines, or other patterns that approximate the shape and contours of the channels. In some embodiments, the article may comprise both an ink and/or adhesive printed layer 320 and backsheet graphics 310, the combination of which work together or separately to accentuate certain channel aspects. Backsheet graphics that approximate the shape and contours of the channels may be arranged such that they fit within the absorbent core area of the absorbent article without extending beyond such area, thereby providing a caregiver a clearer communication of the benefits of the channels.

Further, in some embodiments, the graphics visually signaling the inner channels will not extend beyond the area of the outer channels themselves, and/or the area where the absorbent core is present, and in some embodiments, the graphics visually signaling the outer channels will not extend beyond the area of the outer channels themselves, and/or the area where the absorbent core is present, and in some embodiments, the graphics visually signaling the inner and outer channels will not extend beyond the area of the inner and outer channels themselves, and/or the area where the absorbent core is present.

In addition, the article may comprise other or additional visual cues that signal the absorbent core channels, such as, for example, embossing. Embossing may be done on the topsheet or on the absorbent core, or, in some embodiments, there may be multi-layer embossing of both the topsheet and the absorbent core. For example, thermal embossing may be done over the channels (where there is no superabsorbent polymer) at a small gap setting to enhance the visual presence. Any embossing may be done alone or in combination with printing to help signal the absorbent core channels. Further, any embossing may be done to the topsheet, core, or both, on-line (during assembly of the absorbent article's component parts) or prior to such assembly, e.g. at the site where a supplier of component parts makes the component part itself.

In some embodiments, the signal may comprise a pattern 330 and/or may cover the majority or even all of the absorbent core. In other embodiments, the signal may be disposed only in discrete areas. For example, in some embodiments, a printed layer 320 or backsheet printing 310 may be printed so as to not overlap or intersect with the absorbent core channels. The term “overlap” as used herein means to cover over a part of, or to have an area in common. The term “intersect” as used herein means items that cut across or through each other, or that narrow and merge.

In order to ensure that absorbent article components (including graphic absorbent article components) are properly oriented when attached to other absorbent article components, registration may be used. Registration may include using a system to detect a location on an absorbent article component and to compare the location against a set point (which may be an operator desired or machine set location). The system may adjust placement of the absorbent article component in accordance with said comparison. For example, the location of absorbent core channels and a printed adhesive layer may be detected and a repeat length of the printed adhesive layer may be altered via a length control system (described in U.S. Pat. Nos. 6,444,064 and 6,955,733). Alternatively, a system may be used to detect and control the longitudinal or machine direction position of a component relative to a desired position on the absorbent article wherein the position at which subsequent occurrences of a first component is attached to a second component is altered to ensure the first component is in the desired location. Such can be performed based on multiple detections and the use of an averaging position, deviation from the desired position, or by detecting less frequently than every occurrence. Additionally, a system may detect a first location on a first absorbent article component and a second location on a second absorbent article component, where the first and second locations may be compared relative to each other and against a set point or desired offset position. The system may adjust placement of the first and/or second absorbent article components in accordance with said comparison. Combinations of detection methods may be used. In some embodiments, registration may be used to optimize the visual impression of the printing and the absorbent core channels.

In some embodiments, any printing, whether a printed adhesive layer, a backsheet graphic, or some combination, may match, or substantially match the shape or contour of the absorbent core channels. In some embodiments, the printing will not necessarily match or correlate with the shape or contour of the absorbent core channels, but will otherwise communicate or signal to the consumer the existence and/or benefits of the channels.

In further embodiments, a signal can communicate another functional characteristic 400 of the article including but not limited to size, absorbency, softness, environmental friendliness, configuration for movement/activity of the wearer, water resistance (e.g. swim diapers), extensibility and the like.

### Array of Absorbent Products

Turning to FIG. 5, the present invention is directed to on an array 100 of component absorbent articles 10a, 10b, 10c comprising at least two different articles, preferably at least three articles, wherein at least one of the articles comprises channel-forming areas as described above. The inventors have found that as more offerings of absorbent articles containing channels are made to consumers, consumers are becoming increasingly confused about which products actually contain channels, and of those that do contain channels, which ones are optimal for their particular wearer, be it themselves or a child in their care, etc. The inventors have also found that while indicia on packaging may provide good information to consumer at the point of purchase about which product is optimal for their particular wearer, once the product is removed and separated from the package, this indicia no longer serves that communication function. This is further complicated in situations where the absorbent articles for multiple wearers may be stored together, post removal from their respective packaging, for example, in a day care center, in a hospital, or even in luggage. Upon yet further diligence, the inventors have found that consumers do not always appreciate that channels are present in those products that contain them, again once removed from the packaging. This is often because the channel forming areas are not visible from the exterior of the absorbent article, due to their proximity to the core, and the presence of additional absorbent article components outward of the core, e.g. topsheets, surge layers, and backsheets. In addition, it is difficult to convey to a broad cross section of consumers which features are present within a product because different segments of consumers may respond to different types of signals. For instance, a product geared towards consumers seeking to understand technical aspects of an article, may best utilize signals that are sharply contrasted from the background or other graphics. On the other hand, another product line may be geared towards consumers who are more concerned with maximum performance at the lowest cost, and may best utilize signals that blend in with one another, conveying the feature is present and works in conjunction with remaining aspects of the article. Moreover, consumers showing a preference for a particular product may be more concerned with certain features of an article, and thus a manufacturer may want to highlight said features on that particular product while not focusing on the same feature for different products.

Attempting to solve one of more of the aforementioned problems, among others, the inventors provide an array of absorbent articles where at least one of the articles includes channels, and the different articles within the array have indicia that signal the presence/absence of channels and/or one or more other attributes that may otherwise not be easily noticeable by a consumer viewing the absorbent article apart from its original packaging.

The articles making up the array may differ in one or more of a variety of functional features 400, including size, absorbency, softness, environmental friendliness, configuration for movement/activity of the wearer, water resistance (e.g. swim diapers), and the like. In nonlimiting examples, the articles may differ in size and the larger sized article(s) may comprise greater amounts of absorbent material and/or larger functional features (e.g., the width, length and/or height of channel-forming areas may be greater in a larger sized article than in a smaller sized article). The articles in the array may be part of a line-up or system of absorbent articles that covers a range of different article sizes or other functional features. The array of absorbent articles may be marketed under a common primary designation, usually a registered trademark such as PAMPERS® or LUVS® (both trademarks of the Procter & Gamble Company). The articles of the array may also be marketed under a secondary designation, which may be the same or different for each articles in the array, for example BABY-DRY™ or CRUISERS™.

Arrays according to the present invention may be provided in a variety of ways. For example, as shown in FIGS. 5A-5D, component articles of the array may embody combinations of signals that help communicate functional features of the component article. In some embodiments, component articles in the array may comprise a constant signal 340, meaning on the articles comprising said constant signal, the constant signal appears in a substantially identical manner. Additionally or alternatively, component articles in the array may comprise a varying signal 350, meaning that varying signal is modified between articles in the array. The varying signal may change in dimensions (e.g., channel height, length, width; pattern coverage area), angle or curvature of lines, thickness of lines, pattern, intensity and/or visibility of the signal, color and combinations thereof.

For example, the curved lines in FIG. 5A may be the signal that indicates the presence of channels in the array and may comprise a varying signal 350. The pattern shown in FIGS. 5B-5D may be the signal that communicates absorbency and/or softness and may comprise a constant signal. One component absorbent article in the array (not shown) having no channels may lack the curved lines signal, while another component absorbent article 10a in the array (FIG. 5B) having channels may have both the curved lines signal and the absorbency/softness pattern signal 350a. A third absorbent article 10b in the array (FIG. 5C) having channels and more superabsorbent than the second absorbent article may have a slightly modified curved lines signal 350b and the same absorbency/softness pattern signal 340. A fourth absorbent article 10c in the array (FIG. 5D), having channels and more superabsorbent than the third absorbent article, may have a slightly more modified curved lines signal 350c and the same absorbency/softness pattern signal 340. As such, a consumer would be able to appreciate the presence of channels and also the relative degrees of absorbency/softness across the array. In these examples, the channels signal varies slightly across the array, and the absorbency/softness signal remains constant across the array (for those absorbent articles containing channels). For illustration purposes, these signals are shown as being visible from the body-facing surface of the absorbent article.

In FIGS. 6A-6C, a further example, is shown where articles of the array may embody combinations of signals that help communicate functional features of the component article. For example, the curved lines in FIG. 6A may be the signal that indicates the presence of channels and may comprise a constant signal 340. For example, the pattern shown in FIGS. 6B-6C may be the signal that communicates absorbency and/or softness and may comprise a varying signal. The signals may be formed by any of the means disclosed above, including but not limited to embossing as indicated in FIG. 6B for example. A first absorbent article 10d in the array (FIG. 6A) having channels, but lacking a soft topsheet, may have the curved lines signal, but no softness signal. A second absorbent article 10e in the array (FIG. 6B) having channels and a soft topsheet may have both the curved lines signal and an absorbency/softness pattern signal 350e, while a third absorbent article 10f in the array (FIG. 6C) having channels and a more soft topsheet than the second absorbent article may have the curved lines signal and a slightly modified softness pattern signal 350f. As such, a consumer would be able to appreciate the presence of channels and also the relative degrees of softness across the array. In these examples, the channels signal is constant across the array (for those absorbent articles containing channels) and the softness signal varies slightly across the array. For illustration purposes, these signals are shown as being visible from the body-facing surface of the absorbent article.

In FIGS. 7A-7B, a further example, is shown where articles of the array may embody combinations of signals that help communicate functional features of the component article, as well as the presence and/or absence of channels. For example, the curved lines in FIG. 7A may be the signal that indicates the presence of channels in the absorbent article and may comprise a constant signal 340. The pattern shown in FIG. 7B may be the signal that communicates absorbency and/or softness and may comprise a varying signal 350. A first absorbent article 10g in the array (FIG. 7A) having channels, but lacking a soft topsheet, may have the curved lines signal, but no softness signal. A second absorbent article 10h in the array (FIG. 7B) having channels and a soft topsheet may have both the curved lines signal and an absorbency/softness pattern signal. As such, a consumer would be able to appreciate the presence of channels and also the relative degrees of softness across the array. In these examples, the channels signal is constant across the array (for those absorbent articles containing channels) and the softness signal varies across the array. For illustration purposes, these signals are shown as backsheet graphics 310 being visible from the garment-facing surface of the absorbent article.

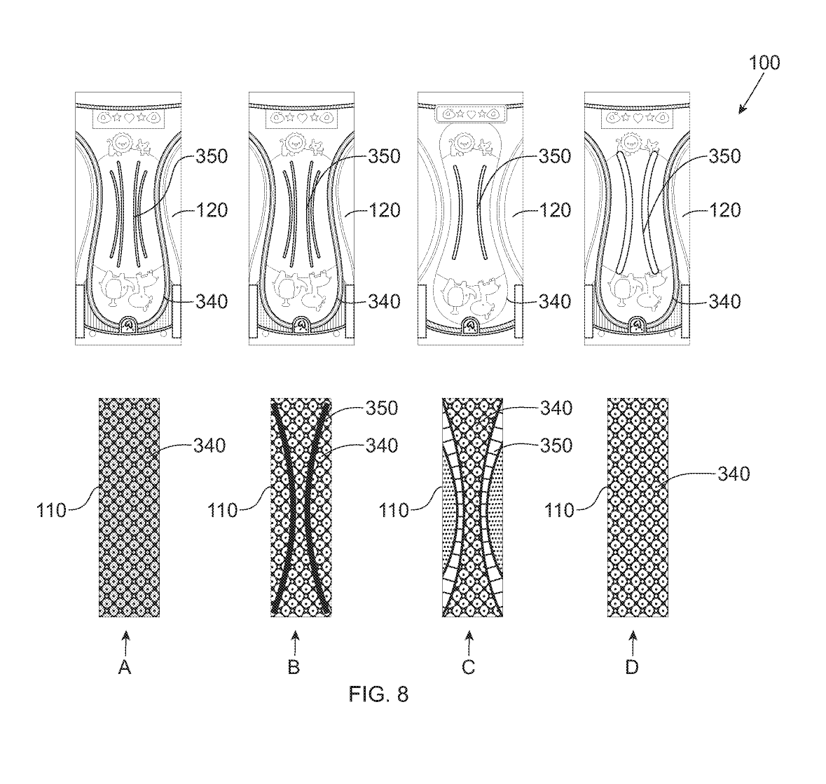

FIG. 8 illustrates component articles A through E in an exemplary array. The component articles have signals visible from the wearer-facing surface and signals visible from the garment-facing surface. The curved lines on the garment-facing surface of articles A-E may indicate the presence of channels in the article and may comprise a varying signal 350 within the array. Notably, more than one article may comprise the same curved lines (e.g., A and B), yet the channel signal may still vary among the remaining articles as shown in FIG. 8. The signals visible from the garment-facing side may also comprise a constant signal 340, such as the U-shaped band that may signal the position of the absorbent core. The signals visible from the wearing facing side may also comprise a constant signal such as the absorbency/softness pattern present in each component article in the array, and a varying signal such as the channel signal that is absent from some articles and present in different forms on other articles. The combination of signals may appeal to different consumer segments and/or provide different messaging regarding article features. Further, one or more signals best communicate to a particular consumer the benefit he or she is looking to experience with the product. Thus, an array having signals targeting particular segments may be better suited to convey desired messaging to a broader audience.

### Test Procedures

The values indicated herein are measured according to the methods indicated herein below, unless specified otherwise. All measurements are performed at 21° C.±2° C. and 50%±20% RH, unless specified otherwise. All samples should be kept at least 24 hours in these conditions to equilibrate before conducting the tests, unless indicated otherwise. All measurements should be reproduced on at least 4 samples and the average value obtained indicated, unless otherwise indicated.

### Centrifuge Retention Capacity (CRC)

The CRC measures the liquid absorbed by the superabsorbent polymer particles for free swelling in excess liquid. The CRC is measured according to EDANA method WSP 241.2-05.

### Dry Absorbent Core Caliper Test

This test may be used to measure the caliper of the absorbent core (before use i.e. without fluid loading) in a standardized manner. This test is described in details in WO2014/093311, incorporated herein by reference.

Absorbent Article Caliper Test

The Absorbent Article Caliper Test can be performed as for the Dry Absorbent Core Caliper Test with the difference that the caliper of the finished absorbent article is measured instead of the caliper of the core. More details for this test are also found in WO2014/093311.

### GENERAL

Any feature or component described herein in relation with one embodiment may be combined with another feature or component of another embodiment unless indicated otherwise.

Dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of absorbent articles, comprising a plurality of absorbent articles, the absorbent articles each having a body-facing side, a garment-facing side and an absorbent core comprising an absorbent material between the body-facing side and the garment-facing side;
wherein the plurality of absorbent article comprises:
at least two absorbent articles of different sizes, and
at least one component article having an absorbent core comprising a pair of channel-forming areas at least partially present in a crotch region of the at least one component article, the at least one component article further comprising a signal communicating the presence of the channel-forming areas, the signal being visible from the exterior of the garment-facing side, the body-facing side, or both.

2. The array of claim 1, wherein the plurality of absorbent articles further comprises an additional component absorbent article, wherein the additional component absorbent article does not comprise channel-forming areas.

3. The array of claim 2 wherein the additional component absorbent article is void of a signal communicating the presence of channel-forming areas.

4. The array of claim 1, wherein the channel-forming areas of the pair of channel-forming areas are symmetrically disposed on opposite sides of the longitudinal axis.

5. The array of claim 1, wherein the at least two component absorbent articles of different sizes each comprise channel-forming areas and wherein at least one dimension of said channel-forming areas increases with an increase in article size.

6. The array of claim 1, wherein the amount of absorbent material in each absorbent core increases with an increase in the article size.

7. The array of claim 1, wherein the absorbent core of each component absorbent article of the array is substantially free of cellulose fibers.

8. The array of claim 1, wherein the absorbent material in each component absorbent article comprises at least about 90% by weight of superabsorbent polymer particles.

9. The array of claim 1, wherein the absorbent material of each component absorbent article is enclosed by a core wrap and the channel-forming areas are formed by attaching a top side of the core wrap to a bottom side of the core wrap along a core wrap bond.

10. An array of absorbent articles, comprising a plurality of component absorbent articles, the component absorbent articles each having a body-facing side, a garment-facing side and an absorbent core comprising an absorbent material between the body-facing side and the garment-facing side;
wherein:
at least one component article comprises a channel-forming area;
at least two component articles comprise a first constant signal communicating the presence of a first functional characteristic; and
the at least two component articles comprise a second varying signal, wherein the second varying signal communicates the presence of a second functional characteristic.

11. The array of claim 10 wherein the second varying signal changes in relation to the degree to which the second functional characteristic is exhibited in the relative component article.

12. The array of claim 10 wherein the first and second functional characteristics are selected from the group consisting of channel-forming areas, size, absorbency, softness, environmental friendliness, configuration for movement/activity of the wearer, water resistance.

13. The array of claim 10 wherein the first functional characteristic comprises the channel-forming area.

14. The array of claim 10 wherein the second functional characteristic comprises the channel-forming area.

15. The array of claim 10 wherein the plurality further comprises an additional component article void of the first constant signal and/or void of the second varying signal.

16. The array of claim 10 wherein first and/or second varying signals are formed by printing, embossing, and combinations thereof.

17. The array of claim 10 wherein the second varying signal varies by one of the group consisting of size, angle or curvature of lines, thickness of lines, pattern, color and combinations thereof.

18. The array of claim 10 wherein the at least two component articles each comprise the first constant signal visible from the body facing side of each article and further comprise an additional constant signal visible from the garment facing side of each article.

19. The array of claim 10 wherein the at least two component articles comprise the second varying signal on the body facing side on each article and an additional varying signal on the garment facing side of each article.

20. The array of claim 10, wherein the absorbent material of each component absorbent article is enclosed by a core wrap and the channel-forming areas are formed by attaching a top side of the core wrap to a bottom side of the core wrap along a core wrap bond.
